(19) United States
(12) Patent Application Publication
(10) Pub. No.: US 2025/0318866 A1
(43) Pub. Date: Oct. 16, 2025

(54) ENDOSCOPIC INSTRUMENT SYSTEM

(71) Applicant: KARL STORZ SE & Co. KG, Tuttlingen (DE)

(72) Inventors: Mathis GARRANDT, Tuttlingen (DE); Klaus M. IRION, Tuttlingen (DE); Sebastian WAGNER, Tuttlingen (DE); Sebastian WENZLER, Tuttlingen (DE)

(73) Assignee: KARL STORZ SE & Co. KG, Tuttlingen (DE)

(21) Appl. No.: 18/866,301

(22) PCT Filed: May 15, 2023
(86) PCT No.: PCT/EP2023/063033

§ 371 Date: Nov. 15, 2024

(30) Foreign Application Priority Data
May 17, 2022 (DE) 10 2022 112 280.8

(51) Int. Cl. A61B 18/14; A61B 17/00; A61B 18/00

(52) U.S. Cl. CPC A61B 18/1445; A61B 17/00234; A61B 18/1487; A61B 2018/00994

(57) ABSTRACT

The invention provides an endoscopic instrument system in which sound, which is generated by RF current coupling into tissue on the distal side, is transmitted in the proximal direction via a structure-borne sound transmission element in order to be converted on the proximal side by an electroacoustic transducer device. The resulting signal can be used in a variety of ways to draw conclusions about the distal situation.

### CROSS-REFERENCE TO RELATED APPLICATIONS

This application is the U.S. national stage of PCT/EP2023/063033 filed on May 15, 2023, which claims priority of German Patent Application No. DE 10 2022 112 280.8 filed on May 17, 2022, the contents of which are incorporated herein.

### TECHNICAL FIELD

The present disclosure relates to an endoscopic instrument system, in particular for minimally invasive surgery on a human body.

### BACKGROUND

In surgery and especially in minimally invasive surgery (MIS), the problem is that an accurate characterization of tissue and especially of deeper tissue structures during an operation is difficult or only possible with great effort with sufficient significance. In MIS, it is also not possible for the surgeon to examine the consistency of a tissue area by direct palpation. They are dependent primarily on the endoscopic image, which shows the surface of the organs or the intracorporeal cavity. Even more difficult is the continuous control of a tissue interaction, such as the treatment of tissue using microwave or radiofrequency (RF) surgery systems consisting of a generator and an instrument that delivers electrical energy to the tissue.

With RF surgery systems, electrically applied energy is spontaneously converted into heat, depending on the mode for cutting, coagulating or welding tissue. It is possible to use intraoperative imaging techniques such as magnetic resonance imaging or X-ray computed tomography to draw conclusions as to the condition of non-visible tissue regions, but this is costly and time-consuming. A real-time method that allows for intraoperative control of an RF interaction is therefore not available.

In addition, there are endoscopically applicable methods such as optical coherence tomography (OCT) or intracorporeal scanners that allow for intracorporeal imaging of tissue behind the endoscopically displayed tissue surfaces. However, these methods are also costly and cannot be used or can only be used to a limited extent for special characterizations and for monitoring, e.g., in thermally induced tissue manipulations, due to the required installation space and the limited, non-specific information.

Frequently performed tissue manipulations include the closure of vessels, for example using bipolar or monopolar radiofrequency instruments that are electrically powered by a radiofrequency generator. It is important to know whether such vascular closures are performed safely and sufficiently firmly and this is crucial for the successful outcome of the operation. There is a requirement and need that, in the case of tissue structures manipulated in this way, quantitative statements can be made about the condition of the tissue during and after the corresponding manipulation.

DE 10 2019 108 140 A1 describes a prior-art bipolar electrosurgical instrument.

US 2010/0168572 A1 describes a system for removing tissue. The system comprises a catheter with an elongate body, which has a radiofrequency electrode for removing the tissue and an acoustic transducer in the distal end region of the elongate body. The acoustic transducer can record acoustic signals. A monitoring unit can record electrical signals from the transducer and interpret them as therapeutic parameters. These parameters can be provided to a user graphically, visually or haptically.

### SUMMARY

The inventors have found that the detection and direct processing of sound inside the human body has a low signal-to-noise ratio. One reason for this has been found to be that electromagnetic fields and signals associated with radiofrequency electrical currents coupled into the body interfere in an undesirable manner with piezoelectric elements used to convert sound into electrical signals.

The present disclosure therefore solves at least the problem of how electrical current can be coupled into the human body with high precision and effectiveness and at the same time precise recordings and conversions of sound can be carried out in order to give a user feedback. In addition, increased design freedom is desired for those elements that are introduced into a patient.

This problem is solved at least by the subject matter of independent claim 1.

Accordingly, an endoscopic instrument system for minimally invasive surgery on a human body is provided, which comprises:

an endoscopic instrument comprising at least:
a distal functional device comprising a (preferably bipolar) electrode arrangement which is designed to couple a radiofrequency electrical current, RF current, into organic tissue (in particular soft tissue) within the human body, and further comprising a structure-borne sound recording element for recording a structure-borne sound response of the organic tissue to the coupled RF current;
a proximal actuating device for handling the instrument system (in particular the endoscopic instrument) and for actuating the functional device from outside the human body;
and
an elongate connecting device which mechanically and functionally connects the functional device to the actuating device, wherein the connecting device comprises a structure-borne sound transmission element which is designed to acoustically transmit the structure-borne sound response recorded by the structure-borne sound recording element in the direction of the actuating device;
and
   an acoustic-electrical transducer device which is designed to be arranged outside the human body when the instrument system is used and which is also designed to detect the structure-borne sound response at the transmission element and to convert it into an electrical response signal.

An endoscopic instrument system is understood to mean, in particular, an instrument system which has a part which is introduced into a human body for treatment or examination. In this context, minimally invasive surgery can mean, for example, that there is no large opening in the body, but that the surgeon operates through a minimal opening.

The terms “distal” and “proximal” are always used from the perspective of the user, i.e., the surgeon or operating physician. Accordingly, the proximal side is a side that is located closer to the surgeon, while the distal side is a side that is oriented away from the surgeon toward the patient. Accordingly, the functional device, which comprises the electrode arrangement, is arranged distally on the instrument, while the actuating device for handling the instrument system is arranged proximally on the instrument.

The endoscopic instrument system according to the disclosure can be operated and/or handled equally by human surgeons and by robots. The proximal side is therefore also the side at which a robot would access the endoscopic instrument system, in particular the proximal actuating device. Whenever reference is made herein to use by a surgeon and/or by a robot, it is understood that the operation and/or handling can also be carried out by the other, unless explicitly or implicitly stated otherwise.

A robot is, for example, a robotic arm which handles and/or operates the endoscopic instrument system in a controlled manner. Handling is understood to mean, in particular, the spatial movement and alignment of the instrument. Operating the instrument comprises in particular triggering, changing or switching off a mechanical, electrical, electronic or other energy-emitting function (e.g., lighting) of the instrument.

The radiofrequency electrical current, RF current, is coupled in intracorporeally and is primarily used to couple in or introduce heat. This in turn is used in particular for tissue fusion, i.e., for example to permanently connect two different tissue parts together. For example, a damaged or malignant tissue part can be surgically removed by a surgeon and the remaining cut edges can then be fused together using the electrode arrangement in order to close the gap created during the operation.

Radiofrequency current is understood to be a current with a frequency of at least 300 kHz. Frequency ranges above 300 kilohertz have the advantage that they trigger no, or fewer, critical nerve stimuli. Tissue fusion can be used, for example, in the context of vessel sealing, i.e., closing an opening in a vessel.

Structure-borne sound is understood here to mean any type of acoustic longitudinal and/or transverse waves propagating in items—including organic tissue—covering a broad frequency spectrum. The frequency spectrum may include sound and/or ultrasound that is audible in particular to humans. Sound propagation within organic soft tissue often resembles sound propagation in liquids rather than sound propagation in conventional solids. The sound transmitted within organic tissue can also be called intra-tissue sound.

A structure-borne sound response of the organic tissue to the coupled RF current is a signal conveyed by structure-borne sound as a carrier, which is generated in the organic tissue as a reaction to the coupled RF current and is transported through it as structure-borne sound. For example, the application of RF energy, i.e., the coupling of RF current into the organic tissue and its spontaneous conversion into heat, can result in the tearing and bursting of cells, cell groups and/or liquid bubbles. The resulting noises (“bursting”) are transmitted, among other things or exclusively, through the organic tissue as structure-borne sound and can thus be recorded by the structure-borne sound recording element as a structure-borne sound response. They therefore provide valuable information about the processes in the organic tissue that are usually not visible to the surgeon.

The structure-borne sound recording element is preferably made of an electrically insulating ceramic material and has an acoustic impedance of preferably between 15*10⁶ and 35*10⁶ [kg/(s*m²)].

The fact that the connecting device is to be of elongate form is to be understood in particular to mean that the apparatus is longer in the longitudinal direction, which extends between the proximal side and the distal side, than in a direction perpendicular thereto. A mechanical connection of the functional device to the actuating device can comprise a structural connection, so that the actuating device and the functional device are arranged in particular rigidly to one another. The mechanical connection may also comprise that a mechanical manipulation, for example of a trigger or handle on the actuating device, is mechanically transmitted to the functional device.

For example, a jaw part of the functional device can be opened or closed by operating a wire pull. A functional connection between the functional device and the actuating device should in particular comprise the fact that a function of the functional device can be triggered, changed or terminated by means of the actuating device. For example, this function may in turn involve opening or closing a jaw part of the functional device, outputting electrical RF current through the electrode arrangement, or the like.

As already explained at the outset, the inventors have found that in the vicinity of the distal functional device, an acoustic-electrical conversion of structure-borne sound signals into electrical signals is subject to considerable disturbance, which is also generated, among other things, by the electromagnetic signals emanating from the electrode arrangement. According to the disclosure, it is therefore provided that the acoustic-electrical transducer device is arranged outside the human body when the instrument system is used, i.e., at a position which is advantageously arranged away from the sources of interference within the human body.

A decisive advantage of this spacing is that thermal and electromagnetic influences or disturbances of the transducer device due to the RF energy coupling in the region of the functional device can be reduced or even completely avoided. This also applies to disturbances due, for example, to mechanical deformations of the organic tissue caused by the functional device, for example by a jaw part, when the organic tissue is compressed.

The spacing of the acoustic-electrical transducer device from the distal working elements, i.e., the functional device and the organic tissue (to be treated), also has the advantage that this results in an extended sound propagation time between the structure-borne sound recording element and the acoustic-electrical transducer device. This means that the structure-borne sound response can be converted more smoothly, for example after the RF current has been switched off.

In particular, the RF current coupled in at the distal end can lead to considerable electrical disturbance in the current technology, since, for example, RF-induced electrical breakdowns, e.g., in the form of arcs, act like electrical jammers. In addition, the arrangement of the transducer device on the proximal part of the endoscopic instrument has the advantage that the transducer device can be designed there with lower requirements and thus greater design freedom and is also easier to access for cleaning and maintenance. In addition, shielding against electromagnetic disturbance can be achieved using a shielding housing.

Usually, the aim is to make the functional device as small as possible so that many different functions can be arranged in a small space. In addition, the functional device comes into constant contact with liquids, heat fluctuations, fluctuations in the electromagnetic field and the like, depending on the application.

Integrating, designing and miniaturizing an electro-acoustic transducer device into the functional device such that it functions perfectly and reproducibly in this environment over the long term is a complex challenge that leads to complex solutions that are, on the one hand, much more error-prone and, on the other hand, substantially more costly.

Therefore, according to the disclosure, the transducer device is arranged outside the human body when the instrument system is used, in particular the instrument, and does not necessarily have to be specially insulated or protected against the aforementioned influences. Even with proximal placement, a certain (lesser) degree of protection, shielding and electrical decoupling is required. However, this protection can be implemented substantially more easily on the proximal side. The electro-acoustic transducer device can be housed in the instrument housing, for example in a handle. The handle is usually already hermetically sealed, on the one hand to protect against external influences and on the other hand for hygienic reasons. Furthermore, the installation space on the proximal side is not limited and therefore no miniaturization is required, meaning that standard (electrical) parts, assemblies and components can be installed (cost-effectively).

The structure-borne sound recording element and/or the structure-borne sound transmission element are designed and configured in particular for recording or transmitting audible sound and/or ultrasound. The inventors have identified the following frequency ranges as particularly preferred frequency ranges for the structure-borne sound to be recorded and transmitted: 1-20 kHz and 20-80 kHz.

The electrical response signal generated by the transducer device can be fed to a variety of applications and/or used to control a variety of functions, as will be described in greater detail below.

The functional device can be designed in particular for coupling RF current into soft tissue, particularly preferably vessels and/or tubular organs. The electrode arrangement for the energy application by coupling in RF current can be designed and configured in particular for vessel sealing by applied electrical monopolar RF energy and/or for vessel sealing by applied electrical bipolar RF energy.

According to some preferred embodiments, variants or developments of embodiments, the functional device has a first jaw part and a second jaw part, which are designed to grip the organic tissue by a relative movement in relation to one another. The first jaw part can be arranged rigidly with respect to the connecting device. Preferably, the structure-borne sound recording element is arranged on this rigidly arranged first jaw part, which has the advantage that the transmission of the structure-borne sound is less disturbed by movements of the first jaw part itself and that the transmission element can be designed comparatively simply. The recording element and the transmission element can be designed in one piece, which leads to optimal structure-borne sound transmission proximally.

For example, the transmission element can preferably be designed as a rigid, elongate element. Alternatively, it is also possible for the structure-borne sound recording element to be arranged on a movable second jaw part or for structure-borne sound recording units of the structure-borne sound recording element to be arranged on both jaw parts. In order to adapt to the movement of the movable jaw part, the transmission element can also be designed to be flexible, articulated, bendable or the like.

If the structure-borne sound recording unit and the structure-borne sound transmission unit are not designed in one part, or cannot be designed in one part due to the construction, the individual units are preferably connected to one another in a form-fitting manner and by appropriate forces that maintain the form-fitting connection even during sound transmission. Such a form-fitting, force-loaded connection could, for example, be a conventional pin-bore-joint connection in the distal mechanics for closing jaw parts, wherein pins and bores are connected to one another in a frictionally engaged and form-fitting manner via tensile forces with which the jaw parts are closed.

Jaw parts are usually referred to as distal-side actuator elements which can be moved relative to each other like jaws or flat-toothed pliers in order to open or close the “jaw” or the “pliers.” In this way, organic tissue, for example, can be grasped and held. This makes it possible to couple the RF current into the organic tissue with high precision, for example to fuse the grasped tissue or the like.

It is particularly preferred if the electrode arrangement is arranged on the first and/or the second jaw part, preferably only on one jaw part, particularly preferably on the rigidly designed first jaw part. In combination with a structure-borne sound recording element arranged on the first jaw part, this ensures that the structure-borne sound response of the organic tissue to the coupled RF current can be tapped as close as possible to where it is induced by coupling the RF current.

Accordingly, according to some preferred embodiments, variants or developments of embodiments, the electrode arrangement is arranged on the first jaw part and/or the second jaw part, in particular on a corresponding inner side of the first jaw part and/or the second jaw part. The inner sides of the jaw parts are those sides of the jaw parts which move towards each other when the jaw parts are closed and may come into contact with each other. Preferably, the structure-borne sound recording element is spatially arranged between a first electrode of the electrode arrangement and a second electrode of the electrode arrangement, wherein the first electrode and the second electrode of the electrode arrangement are particularly preferably arranged on a rigid first jaw part.

The structure-borne sound recording element is thus arranged as close as possible to the point where the RF current is coupled into the organic tissue. The structure-borne sound response can thus be recorded particularly precisely and therefore in particular also has a particularly good signal-to-noise ratio.

The structure-borne sound recording element is generally advantageously arranged in a functional region of the instrument such that, when the instrument is used, it is in contact as directly as possible with the location where sound events originate, i.e., in particular with the tissue to be treated. Some variants of the instrument have a rib on one jaw part in order to generate an increased pressure in a region through which the coupled RF current flows when the jaw parts are pressed together. This creates a region of highly compressed, pressed tissue with a small volume through which the coupled RF current can be conducted. This combination ensures an efficient effect of the RF current. This is particularly advantageous for so-called vessel sealing instruments, which are used to seal vessels by compressing tissue and heating it using the RF current.

In such instruments with a rib, the structure-borne sound recording element can preferably be arranged in the functional surface of the rib, i.e., in or on the surface with which the tissue can be compressed. This surface can also be called a gripping surface. Alternatively or additionally, the structure-borne sound recording element can also be arranged on a surface of a jaw part which is opposite the functional surface of the rib (contact surface) and interacts with it.

The structure-borne sound recording element can be made of an electrically insulating material and/or can be provided with electrical insulation. This is in particular advantageous if the structure-borne sound recording element is arranged such that, when the instrument is used, it is adjacent to a tissue region through which electrical current is passed. Alternatively, the structure-borne sound recording element can also be made of electrically conductive materials (e.g., steel, etc.) or can comprise such materials. In these cases, it is advantageous if a compensation device is arranged on the proximal side of the instrument and is designed to avoid, reduce or compensate for disturbing electrical influences and couplings. For example, the structure-borne sound transmission element can be designed from distal to proximal such that disruptive electrical influences or couplings are avoided, e.g., by the structure-borne sound transmission element being made entirely or partially from non-conductive material and/or being electrically insulated or shielded accordingly.

The structure-borne sound recording element can generally advantageously be designed such that sound events in the tissue are recorded with as little loss as possible (e.g., due to reflections) and transmitted from distal to proximal with as little loss as possible. For this purpose, the sound characteristic impedance (or acoustic impedance) can preferably be selected so that it is as close as possible to that of the tissue to be treated. In some embodiments, a sound characteristic impedance adjustment device may be provided which is designed to adjust the sound characteristic impedance of the structure-borne sound recording element, either automatically or due to a manual setting.

Values between 1.4 Ns/m³ and 104 Ns/m³ have proven to be advantageous values for the sound characteristic impedance of the structure-borne sound recording element, preferably between 20 Ns/m³ and 80 Ns/m³, particularly preferably between Ns/m³ and 60 Ns/m³. The structure-borne sound recording element can be thin and flat, which can be advantageous for recording transverse, longitudinal and additional vibrations (e.g., bending vibrations). Such a thin and flat structure-borne sound recording element can, for example, have wall thicknesses in the range of 0.2 mm to 1.0 mm.

The structure-borne sound recording element can, for example, comprise or consist of one or more of the following materials:
steel (e.g., surgical steel);
ceramic material;
plastics material (in particular with a Shore A value of less than 70, see below);
a reinforced, electrically insulating plastics material comprising glass beads, glass fibers, carbon fibers, quartz, ceramic and/or other similar fillers and/or reinforcing materials;
and/or the like.

According to some preferred embodiments, variants or developments of embodiments, one or both electrodes of the electrode arrangement can also be designed as a structure-borne sound recording element or can form part of a structure-borne sound recording element.

According to some preferred embodiments, variants or developments of embodiments, an entire jaw part can also be designed as a structure-borne sound recording element.

According to some preferred embodiments, variants or developments of embodiments, the transducer device has a piezoelectric element, particularly preferably a piezoelectric element which is arranged in the proximal region such that it is arranged outside the body when the instrument is used.

Piezoelectric elements are particularly well suited to converting acoustic vibrations, be they longitudinal and/or transverse waves, into electrical signals. In principle, all piezoelectric materials known in the prior art can be used. A piezoelectric element can, for example, consist of lead zirconate titanate (PZT) or can contain PZT. Also conceivable are piezoelectric elements made of zinc oxide ceramic, or flexible piezopolymers such as piezoelectric films made of PVDF (polyvinylidene fluoride or polyvinylidene difluoride) and/or composites thereof and/or of PZT.

Piezoelectric fibers, which may contain and/or consist of PVDF nanofibers or PZT fibers, are also possible. The preferred shape and arrangement of the one or more piezoelectric elements depends on the desired application as well as on the properties (frequencies) of the structure-borne sound response and on whether transverse and/or longitudinal vibrations and/or other structure-borne sound waves, such as “bending waves”, are to be detected and converted. Such additional structure-borne sound waves can arise, for example, through the coupling of longitudinal and transverse waves.

PVDF has the advantage of a very high dynamic and frequency range (10⁻⁸ up to 10⁵ N/cm² or 0.1 Hz-1 GHz) and is therefore well suited for detecting low (structure-borne) sound pressures in a broad frequency range. In addition, temperature changes also lead to charge changes in the PVDF film material. It is thus possible to measure the temperature on the distal side using a PVDF element and the structure-borne sound response on the proximal side of the structure-borne sound transmission element using a PVDF element, e.g., a PVDF film. A temperature sensor of the instrument system, in particular of the instrument, can also have a piezo element, in particular a PVDF film.

For converting transverse vibrations, for example, a piezoelectric film arranged concentrically around the connecting device is advantageous. For converting longitudinal vibrations, a piezoelectric element (or other transducer device) attached to a proximal end of the connecting device is advantageous.

According to some preferred embodiments, variants or developments of embodiments, the transducer device comprises an optical detection system, for example based on a time-resolved distance measurement, which is realized by measuring the time of flight of a laser beam in the manner of an “optical microphone”. In this way, a mechanical decoupling between the transducer device and the transmission element can advantageously be provided, which can further increase the design freedom, since the transducer device does not necessarily have to be applied to the transmission element with a perfect fit.

According to some preferred embodiments, variants or developments of embodiments, the transducer device comprises a MEMS sensor, in particular a MEMS accelerometer, i.e., an acceleration measuring device. Such high-precision MEMS accelerometers, which are known from the prior art, can also be used to precisely record and convert the structure-borne sound response transmitted via the transmission element.

According to some preferred embodiments, variants or developments of embodiments, the transducer device is designed to detect structure-borne sound transverse waves and/or to detect structure-borne sound longitudinal waves and/or other structure-borne sound waves on the structure-borne sound transmission element. The structure-borne sound transmission element can have individual sound transmission units, of which, for example, one is designed to transmit structure-borne sound longitudinal waves and/or the next to transmit structure-borne sound transverse waves and/or another to transmit further structure-borne sound waves such as bending waves.

The transducer device can also have two or more transducer units, for example a first transducer unit which is designed and configured to detect structure-borne sound transverse waves and a second transducer unit which is designed to detect structure-borne sound longitudinal waves on the structure-borne sound transmission element and, for example, a further transducer unit for detecting further structure-borne sound waves such as bending waves.

The first, the second and/or several transducer units can be integrated into one another, adjacent to one another or also designed and arranged separately from one another. Since different processes and events within organic tissue can generate different types of structure-borne sound waves, the separate recording of structure-borne sound transverse waves on the one hand and structure-borne sound longitudinal waves on the other hand can provide valuable information about these events and allow for corresponding conclusions to be drawn. A common electrical response signal may be generated, or the electrical response signal may comprise a first electrical response signal generated by electrically converting the structure-borne sound transverse waves and a second electrical response signal generated by electrically converting the structure-borne sound longitudinal waves.

To detect structure-borne sound longitudinal waves, a piezoelectric element of a transducer device can be arranged in a proximal end face or in a cross-sectional area of a proximal region of a shaft of the connecting device or of an internal working element. This can be realized, for example, by using a PZT ceramic disc, a PVDF film or the like.

An internal working element can be understood as, for example, pull and/or push elements for moving distal actuators such as jaw parts, cutting blades, etc. Examples of pull and/or push elements are a tension rod and/or compression rod or a tension rope/wire.

It is also conceivable that a piezoelectric element of a transducer device is integrated into a component for transmitting the electrical current. In a further embodiment, a suction-irrigation tube or the like integrated into the shaft or located inside it can also be used as a structure-borne sound transmission element, and a transducer device can be attached in its proximal region.

In a further embodiment, at least one additional structure-borne sound transmission element located or integrated inside the instrument shaft can serve as wire- or rod-shaped element to which one or more transducer units are attached on the proximal side. This embodiment has the advantage that the structure-borne sound transmission can then be completely decoupled from other (mechanical) functions. This also makes it possible to adapt the wire- or rod-shaped structure-borne sound transmission element(s) in cross section and length to the most important frequencies to be transmitted in order to be able to specifically exploit resonance effects for certain frequency ranges.

According to some preferred embodiments, variants or developments of embodiments, the transducer device is integrated into the endoscopic instrument. This allows for a particularly compact arrangement and easy handling, since there is no need to handle another device or element other than the endoscopic instrument.

According to some preferred embodiments, variants or developments of embodiments, the structure-borne sound transmission element is thin and slim. For example, the structure-borne sound transmission element can have a cross section of 0.05 mm to 10 mm, preferably between 0.2 mm and 5 mm, particularly preferably between 0.3 mm and 0.5 mm.

The structure-borne sound transmission element can be flexibly and/or movably fastened or mounted so that it can vibrate freely both transversely and longitudinally. For example, unnecessary “stiffeners” and/or contacts and/or fastenings between the sound-transmitting element(s) and other components can be deliberately avoided by design.

According to some preferred embodiments, variants or developments of embodiments, a structure-borne sound transmission element made of a relatively harder material can be completely or partially embedded in or surrounded by a relatively softer medium. In this way, the structure-borne sound transmission element can oscillate with as little damping as possible. Preferably, the relatively softer medium has a hardness of 1000 MPa or less (e.g., a thin plastics coating) and/or a Shore A hardness of less than 70 (preferably less than 50, particularly preferably less than 30) and/or a density (in the case of foams) of less than 30 kg/cbm with preferential use of open-pore foams.

According to some preferred embodiments, variants or developments of embodiments, the endoscopic instrument system also comprises a trocar sleeve through which at least the functional device can be passed and introduced into the human body during minimally invasive surgery. The transducer device can advantageously be integrated into the trocar sleeve. An advantage of this is that the endoscopic instrument can be designed more simply. In addition, the appropriately designed trocar sleeve can be used together with a variety of different endoscopic instruments, whereby it only has to be ensured in each case that the structure-borne sound response at the transmission element can be detected by the transducer device integrated in the trocar sleeve.

According to some preferred embodiments, variants or developments of embodiments, the transducer device is attached to a sealing device of the trocar sleeve, which seals a distal space within the trocar sleeve from a proximal space within the trocar sleeve. Naturally, sealing devices are designed and configured such that they fit particularly closely to the endoscopic instrument passed through the trocar sleeve in order to ensure a particularly good seal. This close contact is also advantageous for the transducer device attached to the sealing device to reliably and accurately record the structure-borne sound response transmitted by the structure-borne sound transmission element.

In this case, it is advantageous if the structure-borne sound transmission element is arranged at least partially on an outer side of the endoscopic instrument such that at least the part arranged on the outer side is in contact with the transducer device arranged on the sealing device while the endoscopic instrument is guided through the trocar sleeve.

According to some preferred embodiments, variants or developments of embodiments, the transducer device is mounted on the trocar sleeve such that it is pressed or can be contacted against the structure-borne sound transmission element, while the functional device is guided through the trocar sleeve. As mentioned above, this can be achieved by attaching the transducer device to a sealing device of the trocar sleeve.

According to other variants, a movable mounting can also be achieved, for example, using a spring element such as a coil or leaf spring. An advantage of this in turn is that the contacting of the transducer device onto the structure-borne sound transmission element (or at least part of it) allows for particularly good structure-borne sound transmission between the structure-borne sound transmission element and the transducer device, whereby the response signal can in turn be generated by the transducer device particularly accurately and with a high signal-to-noise ratio.

According to some preferred embodiments, variants or developments of embodiments, the structure-borne sound transmission element comprises a rigid solid body, a hollow waveguide and/or a prestressed wire. These are various implementation options which provide both reliable structure-borne sound transmission and good compatibility with the requirements of an endoscopic instrument system, in particular an endoscopic instrument.

Examples of structure-borne sound transmission elements, in particular for rigid instruments, are additionally:
a rigid, elongate, metal rod;
a rigid, elongate metal push-pull transmission element; and/or
a rigid, elongate instrument shaft (e.g., shaft of the connecting device).

Examples of structure-borne sound transmission elements, in particular for flexible instruments, are additionally:
a flexible wire with pre-tension;
a flexible push-pull transmission element, e.g., implemented in the form of a Bowden cable;
a flexible light guide; and/or
a flexible sound conductor in the form of a hollow waveguide, e.g., a tube filled with a liquid, e.g., water, oil, or a metallic liquid such as a gallium alloy.

According to some preferred embodiments, variants or developments of embodiments, the instrument system further comprises a computing device which is designed to receive the electrical response signal and to generate an output signal at least based thereon. The output signal can be designed in a variety of ways and can, for example, be designed to control an output device such as a screen and/or a loudspeaker.

The output signal can advantageously allow for quantitative tissue characterization and/or monitoring, in particular of manipulated tissue, for example in RF-current-assisted tissue fusion by an endoscopic (RF current) instrument. Such monitoring is particularly advantageous to decide whether the thermally induced closure of a blood vessel is sufficiently stable to withstand the corresponding maximum pressure (greater than the maximum systolic blood pressure). The output signal can therefore generally indicate a quantitative tissue characterization or specifically indicate whether a tissue closure is sufficiently stable.

It has been found that the acoustic information contained in the structure-borne sound response depends, among other things, on the type of tissue (in particular soft tissue), the energy density introduced, the resulting temperature, the liquid content of the tissue and the number of denatured tissue regions or cell groups. Due to the cavitation effects that occur (bang/burst), this acoustic information is not only in the frequency range of human hearing (below 20 kilohertz), but also above it (ultrasound, from 20 kilohertz).

It has also been shown that the more intense the cavitation events (“bursts”) are and the closer they occur to the end of the vessel sealing process, the worse the vessel sealing is classified. Based on an analysis of the structure-borne sound response, the computing device can thus provide a characteristic description of the tissue condition during and shortly after a treatment (i.e., coupling of RF current). The output signal can therefore indicate a current tissue condition of the organic tissue.

The computing device may in particular be a general-purpose computer which is specially configured for this application. The computing device can be fully local, fully remote (e.g., cloud computing platform, remote server) or partially local and partially remote (e.g., local terminal connected to remotely connected server). The computing device can in particular have one or more central processor units (CPUs), one or more graphics processor units (GPUs), a random access memory (RAM), a non-volatile data memory and input and/or output interfaces, all of which are operatively connected to one another.

The computing device may also comprise one or more application-specific integrated circuits (ASICs), field-programmable logic gates (FPGAs), microprocessors and/or the like. The computing device can be realized entirely in hardware, or partly in hardware and partly as software.

According to some preferred embodiments, variants or developments of embodiments, the computing device is designed to generate the output signal in real time. In this way, the output signal can provide a user of the endoscopic instrument system, in particular a surgeon, with valuable real-time feedback about the events in the organic tissue. The instrument system can thus comprise an improved and controlled human-machine interaction, which allows the user to obtain information about internal processes or conditions in the instrument system based on the output signal and to react appropriately thereto.

According to some preferred embodiments, variants or developments of embodiments, the output signal comprises an audible acoustic output signal. Such an acoustic output signal can advantageously indicate various conditions of the organic tissue, into which the electrical RF current has been or is currently coupled, and/or processes therein. The audible acoustic output signal may comprise spoken speech, which may be generated, for example, by a speech generator. The speech can be generated and output in quasi-real time, or after each RF coupling process, for example with the aim of evaluating and/or naming possible problems that have occurred.

However, the audible acoustic output signal can also represent an accurate reproduction or a frequency-shifted reproduction of the structure-borne sound response recorded by the structure-borne sound recording element in a defined frequency range, for example in the ultrasound range, similarly to how a false-color image comprises a frequency-shifted representation, visible to humans, of an electromagnetic spectrum that is actually imperceptible to humans.

As a further alternative, the audible acoustic output signal may also comprise a continuous sound output with different acoustic frequencies audible to humans, each of which indicates a condition or process in the organic tissue, i.e., communicates it to the user. For example, it can be provided that the computing device determines, based on the electrical response signal of the acoustic-electrical transducer device, whether the coupling of the RF current is currently within acceptable parameters or not. If too much heat is coupled in at certain points, the undesirable effects described above can occur.

A pitch (corresponding to a frequency) of the audible acoustic output signal can indicate, discretely or continuously, whether the function currently performed by the functional device is entirely within the acceptable range (e.g., low frequency), entirely within the unacceptable range (e.g., high frequency), or in between (frequency interpolated between the high frequency and the low frequency).

The particular advantage of an audible acoustic output signal is that the user of the endoscopic instrument system usually receives visual feedback on the course of the surgical procedure by observing the patient and the instrument and, if the endoscopic instrument is guided manually, also experiences haptic feedback. An audible acoustic output signal extends these feedbacks to include acoustic feedback, thus providing the user with an additional information channel that they can use without affecting the visual and/or haptic feedback channel.

According to some preferred embodiments, variants or developments of embodiments, the computing device and/or the transducer device are configured such that the output signal is based only on those response signals which are based on a structure-borne sound response which was recorded while no electrical RF current was coupled in. In other words, a measurement of the structure-borne sound response is carried out intermittently (or: in an alternating manner) to a pulsed RF energy application. Switching off the pulsed electrical RF current can initiate (or: trigger) the measurement, i.e., in particular the evaluation of the electrical response signal of the transducer device.

For example, the entire endoscopic instrument system can be designed and configured such that phases of coupling the electrical RF current alternate with phases of evaluating the response signal. In order to coordinate the timing of the coupling and, for example, the evaluation, a signal exchange between a radiofrequency generator of the instrument system and the computing device can be provided. The timing can ensure that the response signal generated by the transducer device is disturbed or distorted as little as possible by the coupling of the electrical RF current.

However, due to the spacing according to the disclosure between the distal structure-borne sound recording element and the proximal transducer device, the signal-to-noise ratio is already excellent in many cases. In these cases, a continuous analysis of the response signal of the transducer device can also be carried out, which in turn allows a continuous metrological control of the surgical procedure (e.g., tissue fusion process). In other words, the analysis of the response signal can thus be continuous.

According to some preferred embodiments, variants or developments of embodiments, the functional device comprises at least one further sensor which is designed to generate at least one sensor signal. The computing device can also be designed to generate the output signal additionally based on the at least one sensor signal. It goes without saying that the output signal can also consist of several partial signals, which are transmitted independently of one another and, under certain circumstances, even evaluated independently of one another. These partial signals are referred to collectively as “the output signal” for the sake of simplicity and description only.

The at least one further sensor can be, for example, a temperature sensor, an electrical impedance sensor, a tissue thickness sensor and/or a pressure force sensor. The generation and evaluation of additional sensor signals has the advantage that the computing device can take into account additional information about the conditions or processes in the organic tissue or also internal conditions or processes in the endoscopic instrument system, in particular the endoscopic instrument itself, when generating the output signal. This can be done, for example, based on predetermined evaluation rules, simulation models, digital twins (for example of the endoscopic instrument) and the like.

One or more of the at least one further sensor may also comprise piezoelectric elements or be designed as such, for example from the materials and material combinations described above. In particular, temperature sensors or sensors for mechanical forces advantageously have piezoelectric elements. Particularly preferably, a sensor can be realized by means of one or more piezoelectric elements which measures one or more forces which are currently exerted on organic tissue by jaw parts of the functional device.

According to some preferred embodiments, variants or developments of embodiments, the computing device is designed to generate the output signal at least partially based on an artificial intelligence entity (AIE). AlEs have proven to be increasingly capable of obtaining complex data sets and, in particular after prior training, drawing conclusions quickly and reliably based on them, sometimes more quickly or taking more contexts into account than would be possible for a human.

The use of an AIE is advantageous in particular if, as described above, at least one additional sensor is provided which generates a sensor signal so that the AIE records as large and diverse a number of input data as possible and generates the output signal based on this. In particular, the AIE should receive the electrical response signal of the acoustic-electrical transducer device as input, wherein further input data are preferably provided, for example temperature data of a temperature sensor, impedance data of an electrical impedance sensor, tissue thickness data of a tissue thickness sensor and/or pressure force data of a pressure force sensor.

According to some preferred embodiments, variants or developments of embodiments, the output signal comprises a control signal which is designed to control a function of the functional device. The output signal can also consist of the control signal. It is thus possible that the output signal, without first being displayed or otherwise made known to a human user (or in addition thereto), directly takes over partial or complete control of the endoscopic instrument.

This can be advantageous in particular if the endoscopic instrument system comprises a robotic device (e.g., a robotic arm) which is designed and configured to handle the endoscopic instrument and to actuate the functional device via the actuating device. If the output signal indicates, for example, that a tissue part is currently being burned, the electrode arrangement can be immediately controlled to reduce the coupled RF current or to switch it off completely. In particular, this can be done faster than a human user would be able to do.

In particular, if the output signal comprises a control signal which is used to control (or: drive) the electrode arrangement, a control loop can be formed: the coupled RF current induces a structure-borne sound response of the organic tissue, which is recorded by the structure-borne sound recording element and transmitted by the structure-borne sound transmission element to the acoustic-electrical transducer device. Based thereon, the transducer device generates an electrical response signal, based on which the computing device generates the output signal which comprises the control signal for the electrode arrangement. Thus, the electrode arrangement can advantageously be controlled in a closed control loop.

The above embodiments and developments can be combined with each other as desired, if appropriate. Further possible embodiments, developments, and implementations of the disclosure also include combinations, which are not explicitly mentioned, of features of the disclosure described above or below with respect to the exemplary embodiments. In particular, a person skilled in the art will also add individual aspects as improvements or additions to the particular basic form of the present disclosure.

### BRIEF DESCRIPTION OF DRAWINGS

The present disclosure is explained in greater detail below with reference to the exemplary embodiments shown in the schematic figures, in which:

FIG. 1 is a schematic representation of an endoscopic instrument system according to an embodiment of the present disclosure;

FIG. 2 is a schematic representation of a functional device of an endoscopic instrument according to an embodiment of the present disclosure;

FIG. 3 is a partial perspective view of the functional device according to FIG. 2;

FIG. 4 is a schematic plan view of a part of the functional device according to FIG. 2;

FIG. 5 is a schematic cross-sectional view of an endoscopic instrument system according to a variant of the instrument system of FIG. 1;

FIG. 6 is a schematic representation of an endoscopic instrument system according to another embodiment of the present disclosure;

FIG. 7 shows schematically a possible variant of the endoscopic instrument system from FIG. 6;

FIG. 8a and FIG. 8b

are schematic longitudinal sectional views through components of an endoscopic instrument system according to a further embodiment of the present disclosure; and

FIG. 9 is a schematic representation of an endoscopic instrument system according to a further variant of an embodiment.

The accompanying figures are intended to provide a further understanding of the embodiments of the disclosure. They illustrate embodiments and, in conjunction with the description, serve to explain principles and concepts of the disclosure. Other embodiments and many of the advantages mentioned are shown in the drawings. The elements in the drawings are not necessarily shown to scale.

In the figures in the drawing, like, functionally like and identically acting elements, features and components are each provided with the same reference signs, unless otherwise specified. The numbering of method steps serves primarily to distinguish them and is not intended to necessarily imply a chronological order, unless explicitly stated otherwise.

### DETAILED DESCRIPTION OF PREFERRED EMBODIMENTS

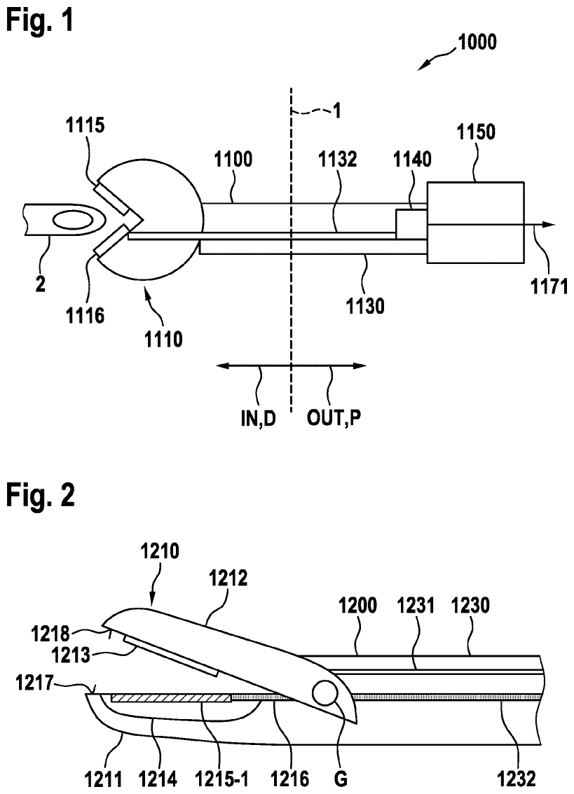

FIG. 1 shows a schematic representation of an endoscopic instrument system 1000 for minimally invasive surgery in a human body according to an embodiment of the present disclosure. The endoscopic instrument system 1000 comprises an endoscopic instrument 1100. The endoscopic instrument 1100 is shown in FIG. 1 as partially introduced into a human body, wherein a boundary of the human body, for example an abdominal wall 1, is shown schematically and in dashed lines. On the left side, marked IN, is the inside of the human body, on the right side, marked OUT, is the outside of the human body. Here, the arrow marked “OUT” points in the proximal direction, P, and the arrow marked “IN” points in the distal direction, D.

The endoscopic instrument 1100 comprises a distal functional device 1110, which in the example of FIG. 1 has thus been introduced into the human body. The distal functional device 110 has an electrode arrangement 1115 which is designed to couple a radiofrequency electrical current, RF current, into organic tissue 2 within the human body. Furthermore, the distal functional device 1110 has a structure-borne sound recording element 1116, which is designed and configured to record a structure-borne sound response of the organic tissue 2 to the coupled RF current.

The endoscopic instrument 1100 also comprises a proximal actuating device 1150, by means of which the instrument 1100 can be handled, be it by a human surgeon and/or by a robotic device such as a surgical robot. The actuating device 1150 can also be designed to actuate the functional device 1110 from outside the human body. This actuation may in particular comprise activating or deactivating the electrode arrangement 1115, moving a mechanical part of the functional device 1110, switching an endoscopic camera of the functional device 1110 on or off and/or the like.

The proximal actuating device 1150 is mechanically and functionally connected to the distal functional device 1110 via an elongate connecting device 1130. Actuations or manipulations of the actuating device 1150 for handling the instrument 1100 and for actuating the functional device 1110 are thus transmitted to the functional device 1110 by means of the connecting device 1130. The transmission can be electrical, mechanical and/or visual, for example. The connecting device 1130 comprises a structure-borne sound transmission element 1132, which is designed and configured to acoustically transmit the structure-borne sound response recorded by the structure-borne sound recording element 1115 in the direction of the actuating device 1150. The structure-borne sound transmission element 1132 is preferably designed for transmission in the frequency range from 20 kHz to 80 kHz and advantageously has particularly low losses in this range compared to airborne sound.

The endoscopic instrument system 1000 also comprises an acoustic-electrical transducer device 1140, which is designed and configured to be arranged outside the human body when the instrument system 1000 is used, and which is also designed and configured to detect the structure-borne sound response at the structure-borne sound transmission element 1132 and to convert it into an electrical response signal 1171.

The conversion can be direct, i.e., a conversion can take place directly from (structure-borne) sound into electrical signals, for example by means of a piezo element. Alternatively or additionally, an indirect conversion may take place, for example via an intermediate medium such as a light beam or laser beam or the like. The transducer device 1140 can be arranged either in or on the endoscopic instrument 1100, as shown by way of example in FIG. 1. However, the transducer device 1140 may also be arranged in another component of the instrument system 1000 or may represent a separate element. In particular, examples will be described below in which the transducer device is part of a trocar sleeve of the instrument system.

The elongate connecting device 1130 preferably has a length of between 15 and 50 cm, particularly preferably between 25 and 40 cm, particularly preferably about 30 cm. The transducer device 1140 is advantageously arranged somewhat further from the electrode arrangement 1115 than the length of the connecting device 1130. The transducer device 1140 is preferably arranged between 32 cm and 52 cm from the electrode arrangement 1115, and particularly preferably arranged between 40 cm and 45 cm away. This helps to ensure that interference or noise signals generated by the functional device 1110, in particular the electrode arrangement 1115, have as little effect as possible on the transducer device 1140 and thus can possibly distort the electrical response signal 1171.

By arranging the transducer device 1140 outside the human body during use of the instrument system 1000, in particular while the endoscopic instrument 1100 is introduced into the human body, the abdominal wall 1 provides additional signal insulation or signal attenuation between the functional device 1110 and the transducer device 140. This increases a signal-to-noise ratio of the signal transmitted by the structure-borne sound transmission element 1132 and realized in the structure-borne sound, i.e., the structure-borne sound response.

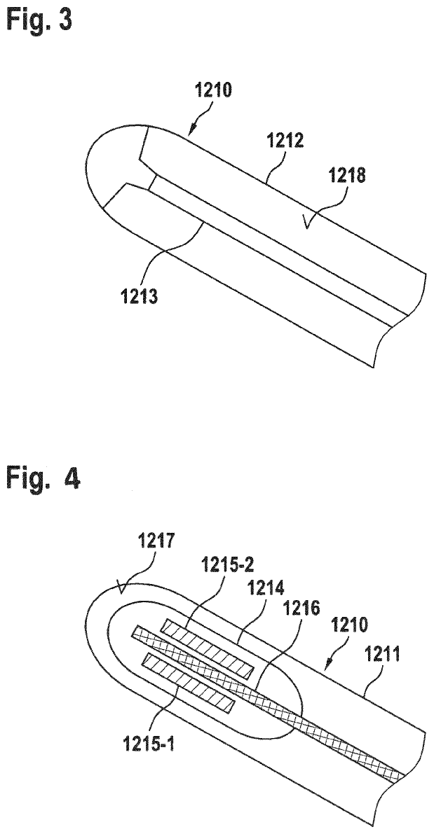

FIG. 2 shows a schematic representation of a distal functional device 1210 of an endoscopic instrument 1200 according to an embodiment of the present disclosure. The functional device 1210 can, for example, be combined with the endoscopic instrument system 1000 and/or the endoscopic instrument 1100 from FIG. 1. While FIG. 2 shows a schematic side view, which partially shows a longitudinal section, FIG. 3 and FIG. 4 are further, partially perspective, views of the functional device 1210.

As can be seen in FIG. 2, the functional device 1210 has a first jaw part 1211 and a second jaw part 1212, which are coupled to one another on the proximal side at the joint G such that the distal ends of the first and second jaw parts 1211, 1212 can move towards and away from one another. In this way, a “jaw” of the functional device 1210 can close (distal ends of the jaw parts 1211, 1212 move towards each other) or open (distal ends of the jaw parts 1211, 1212 move away from each other). With this jaw of the functional device 1210, the organic tissue can thus be gripped, pinched, touched, squeezed or the like, wherein in particular an inner side 1217 of the first jaw part 1211 and an inner side 1218 of the second jaw part 1212 come into contact with the organic tissue and, for example, exert pressure on the organic tissue. The basic structure of such jaw parts 1211, 1212 is in principle known from the prior art.

In the example shown, the part 1211 is designed as a rigid jaw part, while the second jaw part 1212 is designed to be movable relative to the first jaw part 1211 and relative to the connecting device 1230. An actuation of a movement of the second jaw part 1212 relative to the first jaw part 1211 and the connecting device 1230 can, for example, be carried out mechanically by means of a wire 1231 or rod. Alternatively, electrical actuation is also conceivable, for example using an electric motor.

A rib 1213 is arranged on the inner side 1218 of the second jaw part 1212 and is shown in perspective from below in FIG. 3, that is, from the direction of the first jaw part 1211 looking at the inner side 1218 of the second jaw part 1212. Such a rib 1213 can be advantageous in particular, as explained above, for so-called vessel sealing instruments. The rib 1213 is formed, for example, from an electrically insulating material. The rib constricts the organic tissue 2, increases the current density of the RF current in the tissue 2, and focuses the thermal effect in the acoustic reaction induced thereby on this region.

The design of the first jaw part 1211 is shown in plan view in FIG. 4. In the general structure of the first jaw part 1211, an electrical insulator region 1214 is embedded in the inner side 1217 of the first jaw part 1211. FIG. 2 shows the first jaw part 1211 in longitudinal section (longitudinal direction), wherein a longitudinal section through the insulator region 1214 can also be seen. Within the insulator region 1214, a first electrode 1215-1 and a second electrode 1215-2 of an electrode arrangement 1215-i are arranged, separated from one another.

As shown in FIG. 4, these two electrodes 1215-1, 1215-2 can in particular be arranged parallel to one another and be elongate, wherein their longitudinal direction extends along a longitudinal direction of the elongate first and second jaw parts 1211, 1212 and, advantageously, also along a longitudinal direction of the elongate connecting device 1230. By means of electrical lines which are guided through the first jaw part 1211 (not shown in the figures), the electrode arrangement 1215-i can be supplied with radiofrequency electrical current, RF current. The RF current can be generated by a radiofrequency generator of the endoscopic instrument system 1000 outside the patient. This RF current can be coupled into organic tissue that is in contact with the electrodes 1215-1, 1215-2. As already described in detail above, this can be done, for example, for cutting, coagulating, welding, etc. of the organic tissue.

To ensure that the RF current only flows through the organic tissue, the electrodes 1215-1, 1215-2 are arranged in the electrical insulator region 1214. Preferably, the insulator region 1214 is formed from a ceramic material which is embedded in the, for example, metal structure of the first jaw part 1211.

As can be seen particularly well in FIG. 4 and is indicated in FIG. 2 in the cross section through the first jaw part 1211, a structure-borne sound recording element 1216 is arranged on the first jaw part 211 between the electrodes 1215-1, 1215-2 and is introduced and designed to record a structure-borne sound response of the organic tissue 2 to the coupled RF current.

For this purpose, the structure-borne sound recording element 1216 is preferably made of an electrically insulating ceramic material. The structure-borne sound induced or generated in the organic tissue 2 can be recorded by the structure-borne sound recording element 1216, for example as a longitudinal wave and/or as a transverse wave. The structure-borne sound response is transmitted in the proximal direction via a structure-borne sound transmission element 1232, which runs at least partially in the connecting device 1230. As already explained above, the structure-borne sound response is then converted outside the human body by the acoustic-electrical transducer device 1140 into an electrical response signal.

The structure-borne sound recording element 1216 and the structure-borne sound transmission element 1232 can, as shown schematically in FIG. 2 and FIG. 4, be identical to one another and in particular formed in one piece. This has the advantage that there are no edges or gaps in between where structure-borne sound waves could reflect or break. This simple and efficient design is made possible by the fact that the first jaw part 1211 and the elongate connecting device 1230 are rigidly connected to one another, for example also formed in one piece with one another, and the structure-borne sound recording element 1216 is arranged on this first jaw part 1211.

Alternatively, it would also be conceivable that the structure-borne sound recording element 1216 is arranged on the movable second jaw part 1212. In particular in this case, the structure-borne sound transmission element 1232 may comprise a movable, articulated or bendable portion which can adapt to and follow the movement of the second jaw part 1212. However, the structure-borne sound recording element 1216 and the structure-borne sound transmission element 1232 can also be designed differently. As already described above, the structure-borne sound transmission element 1232 can be designed to be flexible or rigid in a variety of ways, or can have both flexible and rigid components or portions.

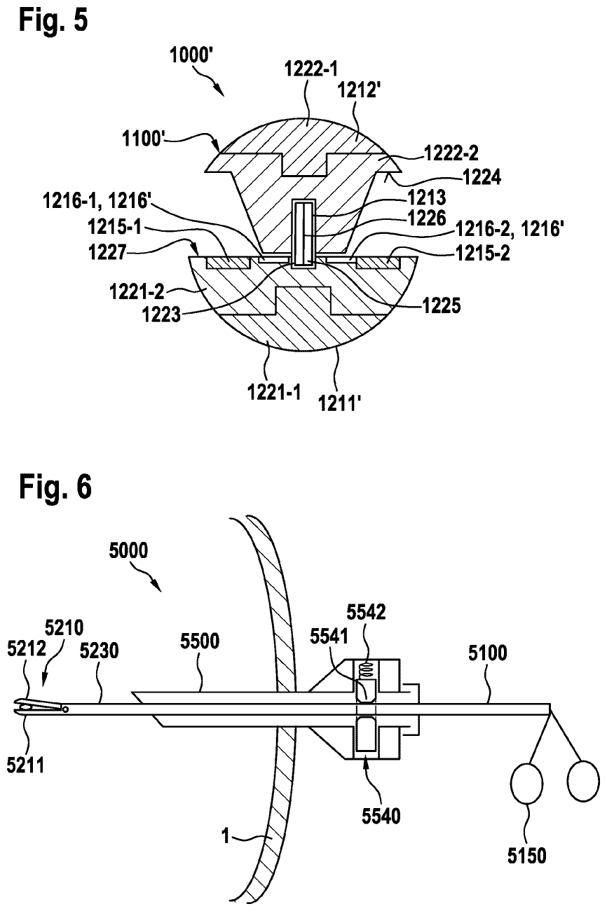

FIG. 5 shows a schematic cross-sectional view of an endoscopic instrument system 1000′ according to a variant of the instrument system from FIG. 1. All details, variations and options described with respect to this instrument system 1000′ are also applicable to the instrument system 1000 of FIGS. 2-4 and vice versa.

In the instrument system 1000′, the endoscopic instrument 1100′ differs from the endoscopic instrument 1100 of the instrument system 1000 in that it includes an integrated scalpel 1225 that is movable along the longitudinal axis of the instrument 1100′ to facilitate the transection of held (or: grasped) and/or sealed tissue. The general arrangement of such a scalpel 1225 is described, for example, in DE 10 2019 108 140 A1, to which reference is hereby made and the contents of which are intended to be fully incorporated by reference in the present application.

Both the first jaw part 1211′ and the second jaw part 1212′ therefore each have a corresponding groove 1223, 1224, which are aligned with each other and within which the scalpel 1225 with cutting edge 1226 is movable, in particular even when the two jaw parts 1211′, 1212′ are completely closed against each other, as shown in FIG. 5. While the scalpel 1225 is fastened to the first jaw part 1211′ and is also actuated from there, the majority of the scalpel 1225 is housed in the groove 1224 of the second jaw part 1212′. Accordingly, the groove 1224 in the second jaw part 1212′ is also larger, preferably more than 50% deeper, particularly preferably more than 100% deeper than the groove 1223 in the first jaw part 1211′.

The first jaw part 1211′ comprises at least two components: a first, externally arranged metal component 1221-1, for example made of surgical steel, and a second, internally arranged (i.e., facing the second jaw part 1212′) electrically insulating component 1221-2, for example made of a ceramic material. In the second component 1221-2, the groove 1223 with the scalpel 1225 is arranged in the inwardly directed surface 1227, and the electrodes 1215-1, 1215-2 are arranged symmetrically on both sides. In between, in each case adjacent to the surfaces of the rib 1213 with which the tissue is gripped or pressed, a structure-borne sound recording portion 1216-1, 1216-2 is arranged between the groove 1223 and one of the electrodes 1215-1, 1215-2. The two structure-borne sound recording portions 1216-1, 1216-2 can be two separate structure-bome sound recording elements or can represent tips of a single structure-borne sound recording element 1216′ split in a fork-like manner on the distal side. This means that the two structure-borne sound recording portions 1216-1, 1216-2 can either each transmit structure-borne sound to a corresponding transducer unit of the transducer device 1140, or a common transducer unit can be provided.

The second jaw part 1212′ also comprises at least two components: a first, externally arranged metal component 1222-1, for example made of surgical steel, and a second, internally arranged (i.e., facing the first jaw part 1211′) electrically insulating component 1222-2, for example made of a ceramic material, which has the rib 1213 with the groove 1224 formed therein. The rib 1213 is also formed symmetrically around the groove 1224 and is shaped such that tissue is gripped between the jaw parts 1211′, 1212′, in particular (or exclusively) where the structure-borne sound recording portions 1216-1, 1216-2 of the structure-borne sound recording element 1216′ rest on the first jaw part 1211′.

Based on FIG. 6, it is very easy to imagine how a tissue is first gripped between the rib 1213 and the surface 1227 and then an RF current is coupled into the grasped tissue between the electrodes 1215-1, 1215-2. In the region of the plateau of the rib 1213, i.e., at its highest elevation, the tissue is thus compressed particularly strongly and a very high current per volume flows, i.e., there is a high current density. As a result, the heat input per volume of the (ungripped) tissue is very high, and thus also the sound generated there, which is ideally recorded by the precisely fitting structure-borne sound recording portions 1216-1, 1216-2. As described above, the sound recording can be improved by a cleverly selected sound characteristic impedance of the structure-borne sound recording portions 1216-1, 1216-2. For example, the structure-borne sound recording portions 1216-1, 1216-2 may be substantially liquid-filled, wherein the liquid is retained at the surface 1227 by a membrane. The structure-borne sound can then be transmitted within the liquid.

FIG. 6 shows a schematic representation of an endoscopic instrument system 5000 according to another embodiment of the present disclosure. The endoscopic instrument system 5000 comprises an endoscopic instrument 5100, which is shown in only a few details in FIG. 6. Unless explicitly or implicitly described otherwise, the endoscopic instrument 5100 may be configured as described above with reference to FIG. 1 to 5. In particular, the instrument 5100 also has a functional device 5210 with a first jaw part 5211 and a second jaw part 5212.

While the functional device 5210 is located at the distal end of the instrument 5100, the actuating device 5150 (again shown in turn schematically here) is arranged at the proximal end of the instrument 5100.

The substantial difference between the endoscopic instrument system 5000 and the endoscopic instrument system 1000 shown in FIG. 1 is that in the instrument system 5000 the acoustic-electrical transducer device 5540 is not integrated into the endoscopic instrument 5100 itself.

Instead, the endoscopic instrument system 5000 comprises the endoscopic instrument 5100 as well as a trocar sleeve 5500. As shown by way of example in FIG. 1, a trocar sleeve 5500 is used to be passed into the human body, here through an abdominal wall 1, in order to create a continuous connection between the inside and outside of the human body, so that the endoscopic instrument 5100 can be introduced through the trocar sleeve 5500 into the human body and can be moved back and forth there. The trocar sleeve allows the instruments to be moved with little friction and, if necessary, quick instrument changes are possible without any problems, i.e., instruments such as scissors can be removed and replaced with, for example, grasping forceps.

In the instrument 5100, the structure-borne sound transmission element (not shown) is designed such that the transmitted structure-borne sound response can be detected, in particular mechanically tapped, on the outer shell of the connecting device 5230. The transducer device 5540 is therefore arranged on the trocar sleeve 5500 and is designed and configured such that it mechanically picks up the structure-borne sound response on the outer shell of the connecting device 5230.

In FIG. 6 it is schematically shown that the transducer device 5540, which can comprise, for example, a piezo element 5541, is mounted on the trocar sleeve 5500 such that it is pressed against the structure-borne sound transmission element, while the functional device 5210 is guided through the trocar sleeve 5500. In this embodiment, the structure-borne sound transmission element can be guided to the outside of the connecting device 5230 in the region in which the transducer device 5540 will be located during proper use. Alternatively or additionally, in this region, the outer shell of the connecting device 5230 itself can be wholly or partially a component of the structure-borne sound transmission element.

The contacting of the transducer device 5540 can, for example, as shown schematically in FIG. 6, be realized by a contact element 5542, for example a spring element such as a coil spring element, by means of which the piezo element 5541 of the transducer device 5540 can be contacted against the structure-borne sound transmission element. In order to prevent the instrument 5100 from resting on one side within the trocar sleeve 5500 due to the contact force, additional contact forces can act on the elongate connecting device 5230 at the same location (in the longitudinal direction along the connecting device 5230) as the transducer device 5540, so that it assumes a spring-loaded central position within the trocar sleeve 5500 if possible.

In the embodiment shown in FIG. 6, the trocar sleeve 5500 has a projection in the radial direction at its proximal end, which not only contains the transducer device 5540, but also represents a gentle closure above the abdominal wall 1.

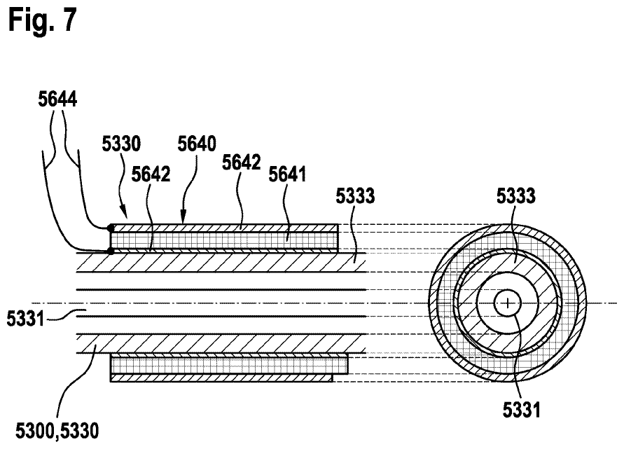

FIG. 7 schematically shows a possible variant of the endoscopic instrument system 5000, wherein an endoscopic instrument 5300 of the instrument system 5000 differs substantially in the design of the acoustic-electrical transducer device 5640 from the transducer device 5540 of FIG. 6.

In FIG. 7, a shaft tube 5333 of a connecting device 5330 of the endoscopic instrument 5300 of the instrument system 5000 is shown. The left part of FIG. 7 shows a longitudinal section through the connecting device 5330, the right part of FIG. 7 shows a corresponding cross-section. A working element or pull element 5331 for mechanical deflection or movement of the distal jaw parts is arranged centrally through the connecting device 5330.

A piezoelectric element 5641 is attached around the coat outer surface of the shaft tube 5333, in particular a PVDF film arranged concentrically with the connecting device 5330, which in turn is concentrically connected on both sides to a corresponding conductive metal layer 5642. These conductive metal layers 5642 or coatings are electrically contacted via electrical lines 5644. Thus, the electrical response signal 1171 of the transducer device 5640 can be transmitted via the electrical lines 5644, for example to a computing device of the instrument system 5000.

As an alternative, the assembly consisting of piezoelectric element 5641 (e.g., PVDF film) arranged (sandwiched) between the conductive metal layers 5642 could instead be arranged on the coat inner surface of a trocar sleeve of the instrument system 5000. The shaft tube 5333 of the connecting device 5330 is then advantageously designed such that it rests against the trocar sleeve as form-fittingly as possible all around.

In both variants, instead of a continuous piezoelectric element 5641, several piezoelectric elements 5641 can be arranged, which can be read separately in order to be able to detect, for example, longitudinal wave propagation or bending waves. The plurality of piezoelectric elements 5641 can, for example, be arranged concentrically and in a matrix-like manner on the coat inner surface or the coat outer surface and can be read in a matrix-like manner. In this way, all wave types of transmitted structure-borne sound can be precisely detected.

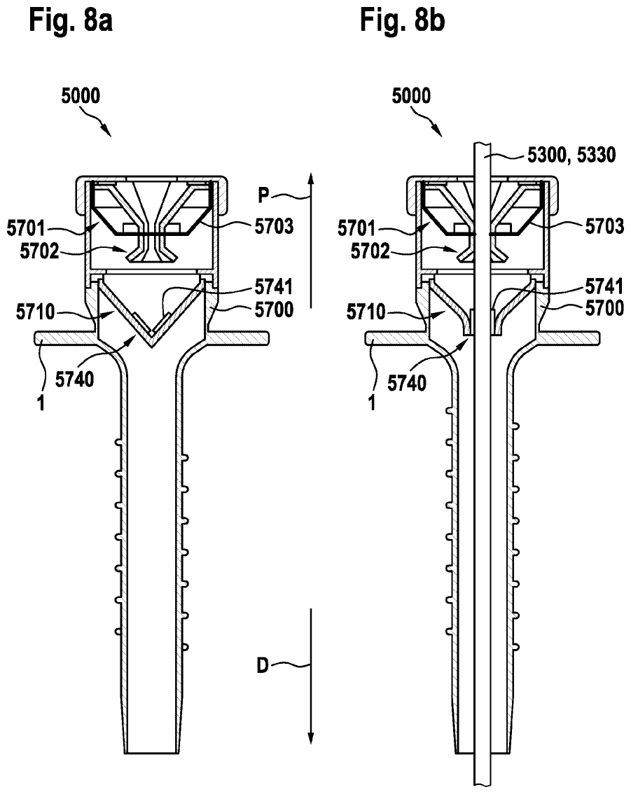

FIG. 8a and FIG. 8b show schematic longitudinal sectional views through components of an endoscopic instrument system 5000 according to another embodiment of the present disclosure. In this embodiment, the instrument system 5000 comprises a trocar sleeve 5700 and an endoscopic instrument 5300.

FIG. 8a shows a situation in which the trocar sleeve 5700 is partially passed through an abdominal wall 1 of a human patient without an endoscopic instrument 5300 being inserted.

FIG. 8b shows the state in which the endoscopic instrument 5300 has been introduced through the trocar sleeve 5700, wherein only the connecting device 5330 is shown schematically. The trocar sleeve 5700 comprises an upper guide device 5701 which guides and supports the insertion of the endoscopic instrument 5300 into the trocar sleeve 5700. Guided by the upper guide device 5701, the connecting device 5330 can thus be guided through an annular seal 5703, as shown in FIG. 8b.

A lower guide device 5702 is arranged in the distal direction D starting from the annular seal 5703. Distally therefrom, in turn, a sealing device 5710 is arranged, which sealingly rests against the connecting device 5330 when the latter is passed through the trocar sleeve 5700. The sealing device 5710 can, for example, be formed from a resilient material and, for example, can comprise silicone or consist of silicone. The sealing device 5710 can, as shown in the substantially rotationally symmetrical longitudinal sectional view, be substantially conical, wherein a cone tip points in the distal direction D and a passage is arranged at the cone tip. When, as shown in FIG. 8b, the endoscopic instrument 5300 is passed through the trocar sleeve 5700, this passage expands due to the resilience of the sealing device 5710 just enough for the sealing device 5710 to lie tightly against the connecting device 5330 all around. The sealing device 5710 may also function as a check valve or be referred to as a check valve.

As also shown in FIG. 8a, one or more piezo elements 5741 can be arranged in the region of the tip of the conical shape of the sealing device 5710, such that when the connecting device 5330 is passed through the trocar sleeve 5700, the piezo elements 5741 rest against the endoscopic instrument 5300 such that structure-borne sound can be detected by means of the piezo elements 5741 on at least part of the structure-borne sound transmission element.

As already described with reference to FIG. 6 and FIG. 7, it is advantageous for this purpose if at least a part of the structure-borne sound transmission element is arranged on the outside of the connecting device 5330, so that the piezo elements 5741 are in direct contact with at least this part of the structure-borne sound transmission element when the endoscopic instrument 5300 is properly introduced into the trocar sleeve 5700. Electrical signals generated by the piezo elements 5741 can thus be output as the response signal of a transducer device 5740, which comprises at least the piezo elements 5741, and optionally further processed via electrical contacts not shown in FIG. 8a and FIG. 8b.

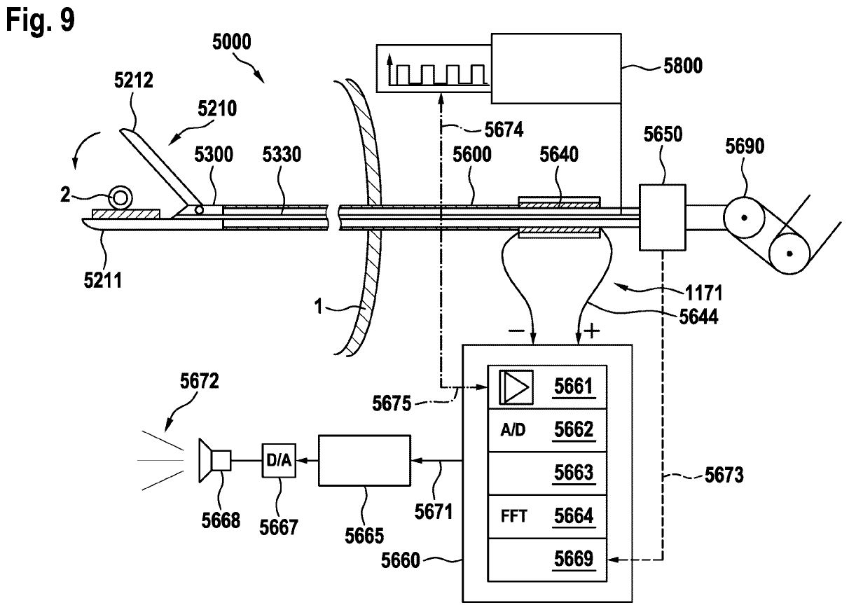

FIG. 9 shows a schematic representation of an endoscopic instrument system 5000 according to another variant of an embodiment. FIG. 9 serves primarily to explain the possible evaluation and further processing of the response signal 1171 by a computing device 5660.

The instrument system 5000 comprises an endoscopic instrument 5300, which can be designed as described above with reference to FIG. 6 and FIG. 7, i.e., that an acoustic-electrical transducer device 5640 is not integrated into the endoscopic instrument 5300, but instead into a trocar sleeve 5600 of the endoscopic instrument system 5000. For example, a robotic arm 5690 is attached as part of a robotic device to an actuating device 5650 for actuating and handling the functional device 5210 of the endoscopic instrument 5300.

The exact embodiment of the endoscopic instrument 5300 and/or the trocar sleeve 5600 is not limited for the description of the embodiment according to FIG. 9. In particular, the endoscopic instrument 5300 can also be designed with an integrated transducer device, the actuating device 5650 can be designed to be guided by a human, the functional device 5210 can be designed with functions other than a jaw part (or additional functions), and/or the like.

In contrast to the illustration in FIG. 7, the transducer device 5640 can also be designed as a resilient PVDF film arranged concentrically on the inner coat surface of the trocar sleeve 5600, layered PVDF films or a bundle of resilient piezoelectric fibers. The resilience ensures that a part of the structure-borne sound transmission element fits particularly tightly to the PVDF film as a piezo element (good “form fit”).

Alternatively, the trocar sleeve 5600 itself can be made of a resilient, highly sound-conducting material, e.g., a fiber composite material, and the transducer device 5640 (or at least a piezo element thereof) can be arranged on the outer coat surface of the trocar sleeve 5600. The structure-borne sound response transmitted by the structure-borne sound transmission element can thus be transported radially outwards through the sound-conducting trocar sleeve 5600 and there in turn converted by the piezo element of the transducer device 5640.

In any case, the electrical response signal 1171 generated by the transducer device 5640 is transmitted to the computing device 5660. This can be done, for example, via electrical lines 5644, but also wirelessly, e.g., via Ethernet, ZigBEE, Bluetooth, LoRaWAN or another wireless signal protocol. The computing device 5660 can be integrated into the endoscopic instrument 5300, the trocar sleeve 5600 or another part of the endoscopic instrument system 5000 or can be arranged in a separate housing. Partial implementation—in addition to local power electronics, for example—by a remotely connected computing device is also possible, such as a cloud computing platform, a server or the like.

In the embodiment shown in FIG. 9, the computing device 5660 comprises an analog amplifier module 5661, an analog-to-digital converter module 5662, an analysis module 5663 and a fast Fourier transform module 5664. These modules can be part of an integrated circuit and/or partially implemented by one or more microprocessors.

When reference is made herein to “modules,” it is to be understood that this does not necessarily mean that such modules are designed as separate units separated from one another.

In cases where modules are realized as software, the modules may be implemented as program code portions or program code components, which may be distinguishable from one another, but which may also be interwoven. Likewise, in cases where one or more modules are realized as hardware, the functions of one or more modules can be implemented by one and the same hardware component.

Alternatively or additionally, different functions of a single module, or different functions of different modules, can be realized on one or more separate hardware components. In this sense, any apparatus, system, method, etc. that has all the properties and functions attributed to a particular module can be understood as having, representing, or implementing such a module. In particular, it may be possible for all modules to be implemented as program code that is realized by a computing device, e.g., a server or a cloud computing platform.

The analysis module 5663 can in particular be designed to generate a control signal 5671 for a frequency converter 5665 based on the response signal 1171 received by the transducer device 5640, amplified by the amplifier module 5661 and amplified by the analog-to-digital converter module 5662. The frequency converter 5665 generates digital frequency signals, which are converted into analog frequency signals by a digital-to-analog converter 5667. These can then be output as an acoustic output signal 5672 through a loudspeaker 5668, preferably in a sound frequency spectrum that is audible to the user of the endoscopic instrument system 5000. Sound waves with a frequency of less than 20 kilohertz (kHz) can be described as audible sound.

Particularly preferred is an output or conversion in a sound frequency range of kilohertz to 80 kilohertz.

As already explained above, such an output signal 5672 (whether acoustic, visual, haptic, etc.) provides the user of the endoscopic instrument system 5000 and in particular the endoscopic instrument 5300 with valuable additional information in quasi-real time. The acoustic output may be an unadulterated or frequency-adjusted output of the structure-borne sound response recorded by the structure-borne sound recording element, or may indicate in an abstract manner a condition or process on or in the functional device 5210.

In a simple variant, the output signal 5672 can have a quiet, comparatively low and constant frequency value when the functional device 5210 interacts with the organic tissue 2 in the desired manner. However, if an undesirable interaction occurs, such as tissue bursting or tearing, this can be indicated by a higher frequency warning tone. The evaluation of the structure-borne sound response and the generation of control signals 5671 based thereon by the analysis module 5663 can be carried out, for example, using rule-based algorithms.

The analysis module 5663 can advantageously analyze the signals digitized by the analog-to-digital transducer 5562 in quasi-real time, for example by counting the acoustic events and their intensity per time interval. Additionally or alternatively, the FFT module (Fast Fourier Transform) can extract frequency-dependent spectral parameters per time interval by means of a (in particular continuous) fast Fourier analysis (FFT). The extracted spectral parameters can in turn be taken into account by the analysis module 5663 when generating the control signal 5671.

It is also conceivable to use an artificial intelligence module, AIEM 5669, which is designed and configured to implement a trained artificial intelligence entity, for example an artificial neural network. Such an artificial intelligence entity receives as input signals signals from the computing device 5660 itself, for example signals in the form of short-term spectra of the fast Fourier transformation module 5664, or also direct signals from the analog-to-digital converter module 5662 and/or the analysis module 5663. The spectral parameters extracted by the FFT module 5564 can also serve as input signals for the artificial intelligence entity.

In addition, it may be advantageous, as already described above, if the endoscopic instrument 5300 preferably in the distal region and/or the trocar sleeve 5600 have additional sensors which generate additional sensor signals 5673. These sensor signals 5673 can thus be used as additional input for the artificial intelligence entity. The additional sensors may be, for example, a temperature sensor, an electrical impedance sensor, a tissue thickness sensor and/or a pressure force sensor or the like. It is particularly advantageous, for example, if a distal-side piezoelectric sensor detects pressure forces on the distal side (e.g., on one of the jaw parts 5211, 5212) and/or the temperature.

The parameters measured or indicated by the sensor signals 5673 are preferably recorded over a time interval. In order for the parameters from the individual time intervals to have statistical significance, the time intervals must be selected with an appropriate duration. In addition, not only the current parameters but also some parameters from previous time intervals can be used advantageously for the analysis. For example, real-time signal processing techniques of continuous averaging with “infinite memory” (equally weighted mean over all time intervals), with “finite memory” (equally weighted mean over a predefined number of time intervals) and/or with “declining memory” (weighted mean in which the time intervals are weighted less and less as the elapsed time increases) can be used.

This can be defined recursively if X(m) is the current running mean of a measured value or parameter in the time interval m and Y(m) is the current measured value in the time interval m:

mean with infinite memory:

$$X ( m ) = X ( m - 1 ) + 1 / m [ Y ( m ) - X ( m - 1 ) 1$$

mean with limited memory:

$$X ( m ) = X ( m - 1 ) + 1 / L [ Y ( m ) - Y ( m - L ) ]$$

mean with declining memory:

$$X ( m ) = X ( m - 1 ) + ( 1 - α ) Y ( m ) , 0 < α < 1.$$

The time intervals of the RF energy application and the time intervals of the pause between are advantageously selected in their duration such that the signal portions therein are quasi-stationary and the parameters derived from them are statistically significant, i.e., in particular, are not too noisy. The time intervals may be longer than the pulse-pause duration of the coupled RF energy in a pulsed RF current injection mode.

From the parameter curves averaged according to one of the averaging methods mentioned above, predictive statements can be made about an ongoing process, e.g., an estimate of the duration until stable vascular closure. Such statements can also be output in the output signal, contained in it, or indicated by it.

In addition to the function of generating an output signal 5672 that is audible, in particular understandable, by the user of the endoscopic instrument system 5000 (or alternatively thereto), the computing device 5660 can also have the function of generating control signals 5674 for one or more components of the endoscopic instrument system 5000. For example, as shown in FIG. 9, the control signals 5674 may control a radiofrequency generator 5800 of the endoscopic instrument system 5000. The radiofrequency generator 5800 serves to generate the electrical RF current, which can be coupled into the organic tissue 2 through the electrode arrangement of the functional device 5210. As shown in FIG. 9, electrical lines for conducting the RF current may run across the connecting device 5330.

Likewise, signals 5675 of the radiofrequency generator 5800 can be received by the computing device 5660 (e.g., at the amplifier module 5661 and/or the analog-to-digital converter module 5662) and taken into account by the computing device 5660 when generating the output signal 5672 or the control signals 5674. In this way, in particular, a control loop can be created.

The signal exchange between the computing device 5660 and the radiofrequency generator 5800 can be used to coordinate the coupling of RF current into the organic tissue 2 on the one hand and the analysis of the structure-borne sound response or the response signal 1171 on the other. As already explained above, this is advantageously done such that periods of coupling the RF current alternate with periods of analyzing the response signal 1171 (with or without a buffer time in between) in order to reduce possible interference of the RF current on the structure-borne sound response.

The control signals 5674 of the computing device 5660 can, for example, also be configured and designed to adjust control variables of the endoscopic instrument system 5000, such as to adjust an electrical power, an electrical voltage, a pulse-pause duration of the coupling of RF current, a pressure force on the organic tissue 2, a position of the jaw parts 5211, 5212 and/or the like.

In the previous detailed description, various features have been summarized in one or more examples to improve the stringency of the presentation. It should be understood, however, that the above description is merely illustrative and not restrictive. It is intended to cover all alternatives, modifications and equivalents of the various features and exemplary embodiments.

Many other examples will be immediately and directly clear to a person skilled in the art on the basis of their technical knowledge in view of the above description. The exemplary embodiments were selected and described in order to best illustrate the principles underlying the disclosure and its possible applications in practice. This allows for those skilled in the art to optimally modify and utilize the disclosure and its various exemplary embodiments in relation to the intended purpose.

1. An endoscopic instrument system for minimally invasive surgery on a human body, comprising:
an endoscopic instrument comprising at least:
a distal functional device comprising an electrode arrangement which is designed to couple a radiofrequency electrical current, RF current, into organic tissue within the human body and further comprising a structure-borne sound recording element for recording a structure-borne sound response of the organic tissue to the coupled RF current;
a proximal actuating device for handling the instrument system, in particular the instrument, and for actuating the functional device from outside the human body; and
an elongate connecting device which mechanically and functionally connects the functional device to the actuating device, wherein the connecting device comprises a structure-borne sound transmission element which is designed to acoustically transmit the structure-borne sound response recorded by the structure-borne sound recording element in the direction of the actuating device; and
an acoustic-electrical transducer device which is designed to be arranged outside the human body when the instrument system is used and which is also designed to detect the structure-borne sound response at the transmission element and to convert it into an electrical response signal.

2. The instrument system according to claim 1, wherein the functional device has a first jaw part and a second jaw part, which are designed to grip the organic tissue by a relative movement in relation to one another, wherein the first jaw part is arranged rigidly with respect to the connecting device and the structure-borne sound recording element is arranged on the first and/or second jaw part.

3. The instrument system according to claim 1, wherein the electrode arrangement is arranged on the first jaw part and/or the second jaw part, in particular on a corresponding inner side of the first jaw part and/or the second jaw part.

4. The instrument system according to claim 1, wherein the structure-borne sound recording element is spatially arranged between a first electrode of the electrode arrangement and a second electrode of the electrode arrangement.

5. The instrument system according to claim 1, wherein the transducer device comprises at least one piezoelectric element.

6. The instrument system according to claim 1, wherein the transducer device comprises an optical detection system and/or a MEMS sensor, in particular a MEMS accelerometer.

7. The instrument system according to claim 1, wherein the transducer device is designed to detect structure-borne sound transverse waves and/or to detect structure-borne sound longitudinal waves on the structure-borne sound transmission element.

8. The instrument system according to claim 1, wherein the transducer device is integrated into the endoscopic instrument.

9. The instrument system according to claim 1, further comprising a trocar sleeve through which at least the functional device can be passed and introduced into the human body during minimally invasive surgery, wherein the transducer device is integrated into the trocar sleeve.

10. The instrument system according to claim 9, wherein the transducer device is attached to a sealing device of the trocar sleeve, which seals a distal space within the trocar sleeve from a proximal space within the trocar sleeve.

11. The instrument system according to claim 9, wherein the transducer device is mounted on the trocar sleeve such that it is pressed against the structure-borne sound transmission element, while the functional device is guided through the trocar sleeve.

12. The instrument system according to claim 1, wherein the structure-borne sound transmission element comprises a rigid solid body, a hollow waveguide and/or a prestressed wire.

13. The instrument system according to claim 1, further comprising a computing device which is designed to receive the electrical response signal and to generate an output signal at least based thereon.

14. The instrument system according to claim 13, wherein the computing device is configured to generate the output signal in real time or quasi-real time.

15. The instrument system according to claim 13, wherein the output signal comprises an audible acoustic output signal indicating various conditions of the organic tissue, into which the electrical RF current was coupled, and/or processes thereon.

16. The instrument system according to claim 13, wherein the computing device and/or the transducer device are configured such that the output signal is based only on those electrical response signals which are based on a structure-borne sound response which was recorded while no electrical RF current was coupled in.

17. The instrument system according to claim 13, wherein the functional device comprises at least one further sensor which is designed to generate at least one sensor signal, and wherein the computing device is designed to additionally generate the output signal based on the at least one sensor signal.

18. The instrument system according to claim 17, wherein the at least one further sensor comprises a temperature sensor, an electrical impedance sensor, a tissue thickness sensor and/or a pressure force sensor.

19. The instrument system according to claim 13, wherein the computing device is configured to generate the output signal at least partially based on an artificial intelligence entity, AIE.

20. The instrument system according to claim 13, wherein the output signal comprises a control signal which is designed to control a function of the functional device.
